(19) United States
(12) Patent Application Publication
(10) Pub. No.: US 2003/0129684 A1
(43) Pub. Date: Jul. 10, 2003

(54) Method for detecting fungicides

(76) Inventors: Rolf Eymann, Reinheim (DE); Heinz-Emil Hauck, Gross-Umstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/311,288

(22) PCT Filed: May 23, 2001
(86) PCT No.: PCT/EP01/05915

(30) Foreign Application Priority Data
Jun. 21, 2000 (DE) 100 29 512.6

(51) Int. Cl. C12Q 1/18

(52) U.S. Cl. 435/32

(57) ABSTRACT

The invention relates to a method for the bioautographic detection of fungicides, in which a developed thin-layer chromatography plate is incubated with a fungal suspension, and zones of inhibition which arise are detected during the fungal growth by fluorescence extinction or by means of optical brighteners.

The invention relates to a bioautographic method for detecting fungicides, in which the growth of fungi is detected directly on a thin-layer chromatography plate (TLC plate) by fluorescence extinction or staining with optical brighteners.

The discovery of novel fungicides is of major importance both in the area of crop protection and for the treatment of fungal diseases in humans. To this end, sensitive, fast and simple detection methods are required. In addition to the search for novel fungicides, it must likewise be ensured by means of effective detection means that, for example, foods, animal feeds and drinking and waste water are free from fungicide residues.

Fungicides are frequently detected by means of thin-layer chromatography. After separation, the fungicides can be identified on the basis of their chemical properties, such as staining behaviour, running behaviour, etc., or by bringing them into contact with fungi. The latter possibility is generally preferred since it also allows fungicides to be detected in complex systems on the basis of their action.

L. Rahalison et al., Phytochem. Anal. 2, pages 199-203, 1991, describe the so-called agar overlay method, in which an agar plate inoculated with fungi is placed on a developed chromatography plate. The fungicides on the chromatography plate diffuse into the agar, where they prevent the growth of and spore production by the fungi.

L. Rahalison et al., Int. J. Pharmacog., 31, pages 68-76, 1993, describe a bioautographic method in which a fungal suspension is applied to the thin-layer plate. After an incubation time of several days, the fungal growth is determined from the spore formation. No staining occurs at the points where fungicides are located since fungi do not grow in these zones of inhibition.

The disadvantage of the known methods is the considerable time requirement. Detection in these methods is carried out via the formation of coloured spores (for example Aspergillus niger forms black spores) or in special cases via the formation of coloured metabolic products (Rhodoturula rubra forms a reddish colour after the growth phase). In both cases, however, incubation, usually for a number of days, is firstly necessary in order to achieve a colour effect.

In addition, the long incubation time frequently results in undesired side effects:

The agar used hitherto is attacked by many fungal strains and liquefies in time. This results, inter alia, in diffusion effects, where the fungicide spots produced by TL chromatography spread.

In the case of relatively long incubation times, overgrowth of fungicide spots can occur, meaning that, in particular, small amounts of fungicide or spots which have spread and diluted due to diffusion can no longer be detected.

The object of the present invention was therefore to develop a method for detecting fungicides which enables rapid and sensitive bioautographic detection.

It has been found that a developed TLC plate can be incubated with a fungal suspension and the growth of the fungi observed after only about 20 hours by means of fluorescence extinction or staining with optical brighteners, and the presence of fungicides can thus be detected.

The present invention therefore relates to a method for the bioautographic detection of fungicides, characterised by the following method steps:

a) provision of a dried, developed TLC plate;

b) application of the fungal suspension to the TLC plate;

c) incubation of the TLC plate;

d) detection of zones of inhibition by means of fluorescence extinction and/or staining with an optical brightener.

In a preferred embodiment, the fungal suspension in, step b) comprises a linear heteropolysaccharide comprising partially O-acetylated glucuronic acid, glucose and rhamnose units.

In a preferred embodiment, the TLC plate is incubated for from 15 to 20 hours in step c).

In a preferred embodiment, the fungal suspension is pre-incubated in step c) before application to the TLC plate.

In a further preferred embodiment, the fluorescence extinction in step d) is detected at 254 nm.

In another preferred embodiment, the fluorescence of an optical brightener in step d) is detected at a wavelength of between 360 and 390 nm.

The present invention also relates to a test kit for carrying out the method according to the invention, which comprises at least one nutrient medium, a linear heteropolysaccharide as gelling agent and a storage-stable test strain for the preparation of the fungal suspension.

In a preferred embodiment, the test kit additionally comprises at least one TLC plate with fluorescence indicator.

In a further referred embodiment, the test kit additionally comprises an optical brightener.

The method according to the invention is generally suitable for detecting fungicides, irrespective of their mode of action. Through the choice of fungal species whose growth is detected, the detection can be directed at certain fungicides. In order to discover novel fungicides against a particular fungal disease, the detection method according to the invention is accordingly generally carried out with the corresponding pathogen or similar fungi. For the general detection of fungicides in, for example, foods or animal feeds, preference is given to the use of fungi from risk group 1, i.e. fungi which do not represent a health risk, such as, for example, Aspergillus niger or preferably Rhodotorula rubra, Saccharomyces boulardii or Saccharomyces cerevisiae.

Firstly, the sample to be analysed for its fungicide content is separated by thin-layer chromatography. A thin-layer chromatographic separation on a TLC plate, i.e. the development of the TLC plate, is carried out under known conditions. For the purposes of the invention, a developed TLC plate is accordingly a TLC plate to which at least one sample has been applied and separated by thin-layer chromatography.

TLC plates having a sorbent layer based on SiO₂ are usually used for this purpose. In rare cases, it is also possible to employ other plates, for example plates coated with cellulose, polyamide or aluminium oxide. Preference is given to the use of silica gel-coated plates which have a diol modification or are water-resistant. Plates of this type have the advantage of being wettable with the aqueous fungal suspension without the risk of damage to or cracking of the silica-gel layer. For detection according to the invention by fluorescence extinction, it is additionally necessary to employ TLC plates with fluorescence indicator, for example HPTLC-FP KG 60, WRF 254s from Merck, Darmstadt.

Suitable mobile phases for example acetonitrile or mixtures of acetonitrile and dichloromethane and a non-polar component, such as n-hexane, are known to the person skilled in the art. The person skilled in the art is furthermore in a position to match the mobile phases to the particular separation problem.

After development, the TLC plate is carefully dried in order to remove residues of the mobile phase.

The developed TLC plate is subsequently incubated with a fungal suspension. This can be carried out, for example, by briefly dipping the TLC plate into the fungal suspension or by spraying with the fungal suspension. In each case, it should be ensured that the fungal suspension is applied uniformly.

It is of course possible for the fungal suspension to be prepared only a defined time before application to the TLC plate. This is typically carried out by mixing a nutrient medium with a form of a test strain. This can be a living culture of the microorganism or a storable form of test strain, i.e., for example, a spore preparation or preferably a lyophilisate or granules. This mixture is preferably pre-incubated for a certain period, usually between 2 and 5 hours. The nutrient medium employed can be any nutrient medium known for the purposes of fungal culture. Preference is given to the use of Sabouraud broth.

In addition, a gelling agent is added to the nutrient medium or the fungal suspension. This preferably comprises gelling linear heteropolysaccharides, which are typically of bacterial origin. Particular preference is given to linear heteropolysaccharides comprising partially O-acetylated glucuronic acid, glucose and rhamnose units, such as Gelrite® from Roth, Germany. Materials of this type form stable gels in the presence of soluble salts. It has been found that this particular type of gelling agent greatly simplifies performance of the test and gives very good results.

On the one hand, a gelling agent comprising linear heteropolysaccharides comprising partially O-acetylated glucuronic acid, glucose and rhamnose units can, in contrast to agar, be processed at room temperature. On the other hand, it has been found that this gelling agent can be sterilised by boiling and sterile-filtering, avoiding complex autoclaving. This is not possible on use of agar as gelling agent. It is particularly important that better detection limits for the detection of fungicides can be achieved with the gelling agent that is preferred in accordance with the invention. It enables very simple and precise adjustment of the flow properties of the fungal suspension, enabling the latter also to penetrate into the pores of the TLC plate. In this way, contact between fungicide and fungi is intensified. Thus, even a small amount of fungicide results in inhibition of the fungal growth.

From 0.08 to 0.5 g, preferably from 0.1 to 0.15 g, of the gelling agent comprising linear heteropolysaccharides comprising partially O-acetylated glucuronic acid, glucose and rhamnose units is typically added to 200 ml of nutrient medium. In the case of too much or too little gelling agent, the fungal suspension can no longer be applied in accordance with the application since it is too stiff or becomes insufficiently solid.

Fungal suspensions for the method according to the invention typically comprise the test strain fungus in a microorganism count of between 10⁵ and 10⁸/ml.

After preparation and, typically, pre-incubation of the mixture of nutrient medium, gelling agent and test strain (for example in the form of a spore suspension), referred to for the purposes of the invention as fungal suspension, the TLC plate is wetted therewith.

The TLC plate is typically incubated at 23-30° C. in a moist environment. The duration of incubation is typically from 13 to 30 hours, preferably from 15 to 24 hours. In most cases, incubation overnight, i.e. for from 15 to 20 hours, is sufficient. In this time, so-called zones of inhibition form at the points of the TLC plate where fungicides are located, since fungal growth is reduced or completely suppressed there. The duration of incubation depends on the number of fungi applied. If incubation is too short, the formation of the zones of inhibition is incomplete due to inadequate growth of the fungi. If incubation is too long, overgrowth of the zones of inhibition can occur.

The zones of inhibition can be detected on the dried TLC plate by means of fluorescence extinction or by staining with an optical brightener. The latter method is somewhat more sensitive.

For detection by means of fluorescence extinction, TLC plates with fluorescence indicator are used. The TLC plates are irradiated at an appropriate wavelength for excitation of the fluorescence. On use of TLC plates with the conventional fluorescence indicator, magnesium tungstate, generally indicated in the product name by the suffix F254s, irradiation is carried out with light of wavelength 254 nm. It has been found that in this procedure, the zones of inhibition become visible as fluorescent spots on the dark background. This simple principle enables detection to be carried out even in the growth phase of the fungi, i.e. before spore formation or the formation of an inherent colour. Whereas the fungal lawn formed results in complete or partial fluorescence extinction, the inherent fluorescence of the TLC plate remains readily visible in the zones of inhibition.

Since the plate is not changed on irradiation, this method is also suitable for analysing the plate a number of times during incubation after various incubation times and following the formation of the zones of inhibition. In rare cases where the fungicide to be investigated itself results in fluorescence extinction, the method of detection by fluorescence extinction can only be used to a limited extent.

In the case of the use of optical brighteners, the TLC plate is, after incubation, sprayed with a stain solution comprising an optical brightener. This stain solution typically comprises from 0.01 to 0.1% (m/m), preferably about 0.04%, of an optical brightener in a basic aqueous solution, for example 0.1 M NaOH solution or 15% potassium hydroxide solution. The optical brighteners used are preferably commercially available brighteners, such as stilbene derivatives, for example disodium 4,4′-bis{(4-anilino-6-morpholino-1,3,5-triazin-2-yl)amino}stilbene-2,2′-disulfonate (C₄₀H₃₈N₁₂O₈S_{2·2}Na), or optical brighteners available under the names Blankophor® (Bayer AG), Calcofluor® White M2R (Sigma), Tinopal® (Sigma) or Leucophor® (Clariant, Canada). Particular preference is given to the use of the optical brightener Blankophor® BA 267% from Bayer AG, Germany. After a development time of 5 minutes at room temperature, the TLC plate is irradiated.

The excitation wavelength is dependent on the optical brightener used. Wavelengths of between 360 and 390 nm are frequently suitable. For Blankophor® BA 267%, an excitation wavelength of 366 nm is highly suitable. It has been found that the optical brighteners exhibit active fluorescence on irradiation in the presence of fungi, whereas no or only slight fluorescence occurs in the case of interaction of the optical brighteners with the fungicides or the TLC plate in the region of the zones of inhibition. The zones of inhibition therefore appear as dark spots on a fluorescent background on irradiation.

In a preferred embodiment, the incubated TLC plate is firstly investigated by means of fluorescence extinction and then, in order further to improve the detection sensitivity, treated with an optical brightener at a suitable point in time and investigated for the fluorescence of the latter.

The detection method according to the invention enables the detection of fungicides to be carried out quickly, sensitively and without major effort.

The use of the preferred linear heteropolysaccharide according to the invention comprising partially O-acetylated glucuronic acid, glucose and rhamnose units as gelling agent simplifies handling since a filtering step can be carried out instead of autoclaving. The flowability of the fungal suspension can easily be varied or set. In this way, the interaction of the fungal suspension with the TLC plate can in turn be optimised.

Detection can be carried out at an earlier point in time by means of the method according to the invention since the zones of inhibition can be depicted with good contrast even in the growth phase of the fungi by means of fluorescence extinction or through the use of optical brighteners.

Fluorescence extinction enables the formation of the zones of inhibition to be followed even during incubation and the incubation to be terminated at a suitable point in time. Staining by means of an optical brightener can subsequently additionally be carried out in order to increase the detection sensitivity. Both staining methods enable for the first time direct detection of fungal growth on TLC plates. Unlike other methods, there is no need to wait until the spores formed can be detected.

For quantitative evaluation, defined amounts of a solution having a known fungicide content can additionally be applied to the plate as reference solution. The fungicide content of the sample can then be calculated via the size of the zones of inhibition.

All essential steps of the method can be carried out in a simple chemical laboratory without particular sterility requirements.

For user-friendly performance of the method, a test kit which can very easily be employed in appropriately equipped laboratories can furthermore be provided in accordance with the invention. The test kit comprises nutrient medium, preferably with the gelling agent preferred in accordance with the invention, preferably in solid form for dissolution in water, a suitable test strain, usually either as lyophilisate, granules or spore preparation, and preferably an optical brightener, typically in solid form or as staining reagent, and optional further constituents, such as suitable TLC plates, reference solutions, etc., which are needed to carry out the method according to the invention.

The test kit furthermore preferably contains a description in which the steps of the method according to the invention are explained.

Even without further comments, it is assumed that a person skilled in the art will be able to utilise the above description in its broadest scope. The preferred embodiments and examples should therefore merely be regarded as descriptive disclosure which is absolutely not limiting in any way.

The complete disclosure content of all applications, patents and publications mentioned above and below, in particular the corresponding application DE 100 29 512, filed on 21.06.2000, is incorporated into this application by way of reference.

### EXAMPLES

1. Detection of TBT (Tributyltin Chloride) in Water

| TLC material: | HPTLC-FP KG 60, Diol F254s, Merck KGaA | |
|---|---|---|
| Fungicides: | TBT | 50 nl application (F1) |
| | sample (25 mg of TBT in 65 ml of double boiled | |
| | deionised water) | application of 200 nl |
| | (F2) and 500 nl (F3) | |

| | |
|---|---|
| Nutrient medium: | Sabouraud 2% glucose broth |
| Gelling agent: | Gelrite ®, Roth |
| Test organism: | Rhodotorula rubra |
| Chromatography: | separation zone 50 mm, eluent acetonitrile, run time 6 |
| | min |

Preparation of the Fungal Suspension:

200 ml of deionised water, 10 g of Sabouraud 2% glucose broth and 0.11 g of Gelrite® are boiled until sterile. The test organism is then added in the form of a lyophilisate or living culture and pre-incubated for 3 hours at 35° C.

Performance of the Detection:

The developed TLC plate is dipped briefly into the fungal suspension, and excess suspension is removed from the base of the plate. The TLC plate is subsequently incubated for 20 hours at room temperature in a can lined with moist paper.

The evaluation is carried out by fluorescence extinction at 254 nm. A zone of inhibition with R_{f} about 1.0 for F1, a zone of inhibition with R_{f} about 1.0 for F2 and F3 and a zone of inhibition on the starting line (R_{f}=0) are evident on the TLC plate.

Result:

| | R_{f} | Zone of inhibition Ø in mm |
|---|---|---|
| F1 | 1 | 6 |
| F2 | 1 | 6 |
| | 0 | 3 |
| F3 | 1 | 7 |
| | 0 | 5 |

The zone of inhibition on the starting line is a degradation product of TBT in water.

2. Detection of Various Fungicides

| | |
|---|---|
| TLC material: | HPTLC-FP KG 60, WRF254s, Merck KGaA |
| Fungicides: | fenpropimorph (A1) |
| | flusilazole (A2) |
| | propiconazole (A3), all from Riedel-de Haen |
| | application 20, 50 and 200 nl in each case |
| Nutrient medium: | Sabouraud 2% glucose broth |
| Gelling agent: | Gelrite ®, Roth |
| Test organism: | Rhodotorula rubra |
| Chromatography: | separation zone 50 mm, eluent acetonitrile/dichloro- |
| | methane/n-hexane (30/40/20 ml), run time 15 min |

Preparation of the Fungal Suspension:

200 ml of deionised water, 10 g of Sabouraud 2% glucose broth and 0.11 g of Gelrite® are boiled until sterile. The test organism is then added in the form of a lyophilisate or living culture and pre-incubated for 6 hours at room temperature.

Performance of the Detection:

The developed TLC plate is dipped briefly into the fungal suspension, and excess suspension is removed from the base of the plate. The TLC plate is subsequently incubated for 20 hours at room temperature in a can lined with moist paper.

The evaluation is carried out by staining with optical brightener (0.04% of Blankophor® BA 267% from Bayer AG, Germany, in 0.1 mol/l NaOH solution). To this end, the TLC plate is dried, wetted with the stain solution and subsequently stored for 5 minutes at room temperature. The evaluation is carried out at 366 nm. The zones of inhibition are evident as dark spots against a fluorescent background.

A zone of inhibition is evident on the TLC plate for each of A1 to A3 in all three concentrations.

| | | Zone of | Zone of | Zone of | |
|---|---|---|---|---|---|
| | | inhibition | inhibition | inhibition | |
| | R_{f} | 2 ppb | 500 ppt | 100 ppt | Detection limit |
| A1 | 0 | ++ | − | − | 2 ppb (2 μg/kg) |
| A2 | 0.28 | ++ | + | − | 500 ppt (500 ng/kg) |
| A3 | 0.48 | ++ | + | − | 500 ppt (500 ng/kg) |

(Key:
− = no zone of inhibition;
+ = small zone of inhibition;
++ = clearly visible zone of inhibition)

1. Method for the bioautographic detection of fungicides, characterised by the following method steps:
a) provision of a dried, developed TLC plate;
b) application of the fungal suspension to the TLC plate;
c) incubation of the TLC plate;
d) detection of zones of inhibition by means of fluorescence extinction and/or staining with an optical brightener.

2. Method for the bioautographic detection of fungicides according to claim 1, characterised in that the fungal suspension in step b) comprises a linear heteropolysaccharide comprising partially O-acetylated glucuronic acid, glucose and rhamnose units as gelling agent.

3. Method for the bioautographic detection of fungicides according to one of claims 1 and 2, characterised in that the TLC plate is incubated for from 15 to 20 hours in step c).

4. Method for the bioautographic detection of fungicides according to one or more of claims 1 to 3, characterised in that the fungal suspension is pre-incubated in step b) before application to the TLC plate.

5. Method for the bioautographic detection of fungicides according to one or more of claims 1 to 4, characterised in that the fluorescence extinction in step d) is detected at 254 nm.

6. Method for the bioautographic detection of fungicides according to one or more of claims 1 to 4, characterised in that the fluorescence of an optical brightener in step d) is detected at a wavelength of between 360 and 390 nm.

7. Test kit for carrying out the method according to one or more of claims 1 to 6, at least comprising a nutrient medium, a linear heteropolysaccharide as gelling agent and a storage-stable test strain for the preparation of the fungal suspension.

8. Test kit according to claim 7, characterised in that it additionally comprises at least one TLC plate with fluorescence indicator.

9. Test kit according to one of claims 7 and 8, characterised in that it additionally comprises an optical brightener.
